(19) United States
(12) Patent Application Publication
(10) Pub. No.: US 2024/0402154 A1
(43) Pub. Date: Dec. 5, 2024

(54) LONG TERM POTENCY ASSAYS FOR CELL AND GENE-MODIFIED CELL THERAPY

(71) Applicant: Wilson Wolf Manufacturing, New Brighton, MN (US)

(72) Inventors: Eric A. Smith, Sugarland, TX (US); Andrew H. Lewis, Houston, TX (US)

(21) Appl. No.: 18/731,199

(22) Filed: May 31, 2024

Related U.S. Application Data
(60) Provisional application No. 63/470,113, filed on May 31, 2023.

(51) Int. Cl. G01N 33/50

(52) U.S. Cl. CPC G01N 33/5011; G01N 33/505; G01N 2500/10

(57) ABSTRACT

Gas permeable devices and methods are disclosed for cell culture, including cell culture devices and methods that contain medium at heights, and gas permeable surface area to medium volume ratios, that do not conform to conventional wisdom. These novel devices and methods allow improvement in cell culture efficiency and scale up efficiency.

### RELATED APPLICATION

The present application claims priority to U.S. Provisional Application No. 63/470,113 filed May 31, 2023, which is hereby incorporated by reference in its entirety.

### TECHNICAL FIELD

The technical field of the invention relates to methods and devices that improve potency assessment and characterization of cells that are manufactured in the field of cell and gene modified cell therapy.

### BACKGROUND OF THE INVENTION

Key attributes of cell products, such as the capacity to kill cancer cells, are assessed in conditions that are more representative of the state that cells will experience in use than existing methods and devices allow. Advantages are obtained through use of devices that provide sufficient oxygen and nutrients for long term assessment such as the ability of the cell product to find and kill cancer cells. When compared to current assessment methods, a variety of benefits accrue, including assessment of thawed cell products without modification to the formulation in which the cell products are cryopreserved. This allows a more representative evaluation of the actual drug product that will be used on patients. Additionally the methods and devices allow for increased assay duration for long-term monitoring of cell product attributes such as cell migration, killing capacity, expansion capacity, and persistence potential without significant operator intervention.

### DISCUSSION OF LIMITATIONS OF CONVENTIONAL TECHNOLOGIES DESCRIBED IN RELATED ART

Cell and gene modified cell therapy (CGT) is a promising area of drug development where the immune system of a diseased patient can be enhanced with cells that attack the disease. CGT may include T-Cells, Tumor Infiltrating Lymphocytes (TILs), Cytokine Induced Killer Cells (CIKs), Natural Killer (NK) cells, B-Cells, Neutrophils, Hematopoietic Stem Cells (HSC's),

Induced Pluripotent Stem Cells (iPSCs), or a combination thereof, where the source material is obtained from a patient or a donor, modified outside the body, expanded to quantities necessary for therapeutic application, formulated, and delivered to a clinical setting for infusion into a patient. These therapies are also sometimes referred to as Advanced Therapy Medicinal Products (ATMPs). Like conventional pharmaceuticals, approval of an ATMP is obtained by establishing benefits over standard of care in terms of safety and efficacy through extensive clinical studies.

Unlike conventional pharmaceuticals, the starting material of CGT manufacturing processes consists of highly variable cellular material. Each patient's or donor's cells have unique cellular characteristics stemming from inherent genetic and lifestyle differences, and in the case of diseased donor's, their diverse prior treatment histories. These cellular differences lead to wider variability within CGT manufacturing processes in comparison to traditional pharmaceutical manufacturing processes. Because the final Cell product is a complex cellular composition, the defining characteristics of CGT drugs are less precise and more difficult to measure and control than traditional pharmaceutical drug product characteristics. For this reason, the final Cell product is defined in large part by the manufacturing process used to make it. “The process is the product” is a phrase that is commonly linked to the FDA viewpoint of Cell products.

Accordingly, CGT drug developers must characterize their manufacturing process and implement effective control strategies that ensure the manufacturing process can repeatably produce the required number of therapeutic cells with the same or similar defining characteristics, independent of the unique characteristics of the starting cellular material. By properly defining and controlling the manufacturing process to achieve that goal, data obtained from patients treated in clinical trials will be inclusive of the variability in the starting cell characteristics from donor to donor, or patient to patient, thereby allowing the CGT drug developer and regulatory agencies to reliably associate clinical outcomes with the manufacturing process used to produce the drug.

In order to characterize manufacturing processes, CGT drug developers identify the final quality targets or specifications that the drug developer believes will contribute to the success of the drug product based on prior experience, existing pre-clinical data, published data, and similar products in the field. This is often referred to as the Quality Target Product Profile (QTPP). The QTPP is a prospective summary of the quality characteristics of a drug product that need to be achieved in order to ensure the desired quality, safety, and efficacy of a cell product. The QTPP can be thought of as a high-level design criterion (sometimes referred to in engineering fields as “Design Requirements” or “Product Requirements”) for a final cell product. QTPP can include, among others, the following attributes: therapeutic indication, shelf life, storage conditions, dosage form, dose regimen, pre-treatment regimen, as well as the safety, identify, purity, and potency characteristics of the final drug product. Developers then identify cellular product quality attributes that may relate to the safety, identity, purity, potency, efficacy, or stability of the drug product. Through risk assessment processes, these quality attributes are further distilled into Critical Quality Attributes (CQAs) which are used by CGT drug developers/manufacturers to define their final drug product specifications. CQAs are associated with the clinical safety and efficacy data obtained during extensive clinical studies, and therefore are also used for demonstrating comparability when manufacturing process or facility changes are implemented. Examples of typical CQAs include, among others: Sterility, Mycoplasma, Endotoxin, Vector Copy Number, Dose range (transduced cells per dose or per body weight), cell concentration, % transduced cells, % CD3+ cells, Viability, cytotoxicity, cytokine release specifications (IFN-g), HLA match, Replication Competent viral vector, CTL activity vs target bearing line, etc.

CQA's require the development and/or implementation of tests that reliably and consistently measure CQAs during manufacturing (in process testing) and at the end of production prior to release (lot release testing). The complex nature of the mechanism of action of CGT drug products makes the development of objective standard assays increasingly difficult. These tests are critical to ensuring that a manufacturing process is producing a therapy that falls within the specified ranges that have proven safe and effective in the clinic. One of the most critical quality control tests is the potency assay. Potency testing is a quantitative measure of the biological activity of a CGT product and establishes that a product meets minimum levels of potency to achieve the desired clinical effect as well as ensures that potency is consistent from manufacturing lot to manufacturing lot. The FDA defines Potency as “the specific ability or capacity of the product, as indicated by appropriate laboratory tests or by adequately controlled clinical data obtained through administration of the product in the manner intended, to effect a given result.” Regulations require that “tests for potency shall consist of either in vitro or in vivo tests, or both.”

FDA guidance suggests that the ideal measure of potency will measure the Mechanism of Action, but because CGT drug products are often complex compositions of cellular material, the Mechanisms of Action are not always fully characterized. Potency is often defined by the CGT product's ability to kill target cells, known as their cytotoxicity. Cytotoxicity is determined by measuring target cell death, which can be assessed by measuring specific markers of cell death or by measuring a reporter protein expressed by the target cell (e.g. luciferase, GFP). Often, a reporter protein expressed within target cells is used for real time assessment of cell therapy product cytotoxicity. However, current methods for this assessment are limited in their assessment of cell therapy product potency because the product material used for the potency testing must be taken prior to final formulation and cryopreservation for clinical administration. In other words, the potency measurement does not measure the final drug product formulation that is eventually administered in the clinic.

Further, potency testing can be performed by non-biological analytical assays such as flow cytometry enzyme-linked immunosorbent assay, reverse transcription polymerase chain reaction, quantitative polymerase chain reaction, micro array, protein binding or enzymatic reactions. If non-biological assays are used, the results of these measurements can only be considered surrogate measures of potency, and thus must be substantiated by correlation to product specific biological activity. The devices and methods described herein can be used as a single biological potency assay or used to substantiate use of surrogate potency measurements and/or be used within an assay matrix, which includes both biological assays and non-biological assays. Skilled artisans familiar with CGT manufacturing recognize the critical importance of developing potency assays early in CGT drug product development so that pre-clinical in-vivo animal data, as well as early stage clinical and late-stage clinical data can be correlated with the results of potency testing. Thorough product characterization will allow repeatable manufacturing and an ability to correlate Critical Quality Attributes, including potency characteristics, with successful outcomes in the clinic.

Potency Assays should eliminate sources of variability as best as possible. This can be achieved by the use of high-quality reagents that have been qualified for use, equipment that is calibrated, and adequate training of operators. Further, simple and robust standard operating procedures as well as the use of an assay that requires minimal operator intervention will eliminate many sources of variability. There exists a need for a robust potency assay devices and methods that eliminate variability introduced by operator intervention and allows longer term evaluation of product.

Additionally, Potency Assays should allow the inclusion of appropriate control conditions that are product specific and should allow the control conditions to be run in parallel with the CGT product potency assay. It can be beneficial to develop a well characterized clinical lot reference material to be used in control conditions. Skilled artisans recognize that other reference materials such as CAR positive cells without co-stimulatory domains as described herein may be beneficial. Reference materials will often be CGT drug product specific. During product development, it can also be beneficial to evaluate different cellular product candidates. For instance, a well-developed longer-term potency assay can be used to evaluate the benefits of one product candidate over another in a longer-term assay. Similarly, the methods and devices described herein will allow for simple comparability studies to assess manufacturing process changes, qualification of new media or other reagents, and assessment of lot to lot consistency in a simple, repeatable, and robust manner.

There exists a need for a simple standardized potency assay that can allow long term co-culture of thawed cell product without reformulation, with target cells, to allow the long-term characterization of cell product cytotoxicity. Cell therapy products are often cryopreserved at a high concentration (e.g., 10×10⁶ cells/mL) using a cryoprotectant, such as 10% DMSO, to maintain viability during storage in liquid nitrogen. DMSO concentrations as low as 2.5% have a negative effect on lymphocyte function (PMID: 29125561), making it difficult to properly assess their cytotoxicity. Additionally, current methods rely on small volumes in which to the perform the assay (e.g., 100 μL in a 96-well plate or 0.3125 mL per cm2), requiring small numbers of cells (e.g., 50,000) so as not to deplete the nutrients of culture media prior to completion of the potency assay. Therefore, current methods for assessing the cytotoxicity of cell therapy products require that the cryopreserved product be washed of cryoprotectant, converting the cell product to a formulation that is amenable to the specific assay being used (PMID: 30910382). Furthermore, current potency assays that allow real-time, long term measurements of cytotoxicity against targets often require product cells to be washed of cryoprotectant and further cultured prior to use in the assay (PMID: 31789308). The relatively small numbers of product cells (e.g. 50,000) being used in current potency assays further limits downstream characterization, such as flow cytometry, where larger numbers of cells are required to characterize smaller subpopulations contained within cell therapy products.

Notably, the prior art does not evaluate the cryopreserved Cell Product (the manufactured cell composition resulting from a CGT manufacturing process) without first washing the cells of the cryopreservant, allowing a period of time for the cells to recover from cryopreservation, re-suspending the cells in the reagent used for the assay, and then proceeding with the assay. Washing the cells is a time consuming process of separating the cells from the cryoprotectant, typically involving centrifugation or filtration. There exists a need for the ability to evaluate Cell Products as they are formulated and in a method that more closely reflects how the product is infused in the clinic. Cell Products are often thawed at 37° C. and subsequently infused without washing the cryoprotectant from the formulation, therefore, a potency assay that can be performed without washing and without a change in the formulation of the Cell Product, will more closely evaluate the Drug Product as it is formulated allowing for more direct Cell Product/Drug Product measurement.

T cells are complex biologics that require multiple functions to kill cancer cells and the CGT field is burdened by highly limited tools for the assessment of complex CGT drug products.

For example, T cells are able to sense chemokine gradients and migrate in the direction of high chemokine concentrations to arrive at tumor sites. Additionally, once at a tumor site, T cells need to navigate and infiltrate the physical environment surrounding and within tumors. Each assay used to study these functions can only assess one T cell function and only do that over a short timeframe. For example, the transwell assay is an elegant system with which to study and assay T cell migratory and infiltrative abilities, but this assay falls short in addressing T cell anti-tumor activity (killing), proliferation, or persistence. Conventional assays to assess T cell killing take many forms: ⁵¹Chromium release, bioluminescence-based, and flow cytometry-based coculture assays measure T cell cytolytic ability, but these assays force colocalization of T cells and target cells, thereby removing the requirement and assessment of T cell migration or infiltration, a key characteristic of T cells in vivo. Furthermore, in order to provide long-term, durable clinical responses, T cells must expand and persist in the tumor microenvironment. Standard in vitro assays used to assess T cell expansion and persistence are performed under controlled settings in the absence of active tumor growth, which limits the correlation of these data to in vivo conditions.

The Table 1 below provides a summary of existing assay types, the functions assessed, and the time that each assay can be performed without manipulation of the assay conditions. There exists a need for methods and devices that overcome the limitations of the prior art and facilitate the ability to perform long-term co-culture assays that evaluate a CGT drug product's ability to expand and persist in the presence of actively growing target tumor cells.

TABLE 1

| | Short Term | | Limited | |
|---|---|---|---|---|
| | Assessment | | Assessment | The Drug Product must be significantly |
| Assay Type | Duration | | Capability | altered prior to assessment |
| Transwell | 12 | hours | Migration | Cells must be removed from cryoprotectant |
| Transwell | 48 | hours | Infiltration | Cells must be removed from cryoprotectant |
| ⁵¹Cr release | 6 | hours | Anti-tumor activity | Cells must be removed from cryoprotectant |
| | | | (killing) | |
| Annexin V | 24 | hours | Anti-tumor activity | Cells must be removed from cryoprotectant |
| (flow cytometry) | | | (killing) | |
| Bioluminescence | 24 | hours | Anti-tumor activity | Cells must be removed from cryoprotectant |
| | | | (killing) | |
| Multiplexed live | 48-72 | hours | Anti-tumor activity | Cells must be removed from cryoprotectant |
| cell analysis | | | (killing) | |
| CFSE | 48-72 | hours | Proliferation | Cells must be removed from cryoprotectant |
| Thymidine | 24 | hours | Proliferation | Cells must be removed from cryoprotectant |
| (3H-Thy/BrdU) | | | | |

Perhaps the closest prior art is an abstract (PMID: 24819215) that provides a high-level overview of methods and devices that facilitate the long-term characterization of T cell product interactions with an in vitro 3D tumor model. The abstract describes inventions disclosed in U.S. Pat. No. 11,613,725. The methods disclosed include a six chamber “maze” that evaluates T cell migration to a chemokine signal created by cancer cells engrafted in a bio scaffold. The bio scaffold is engrafted with cancer cells in a separate culture system, and then the bio scaffold with engrafted cancer cells is removed from the culture system and placed in the assay device. T cells are then placed in a culture compartment that is different from the culture compartment containing the bio scaffold with engrafted cancer cells. The assay device includes migratory pathways, or opening between separate culture compartments that allow the T cells to migrate through a “maze” within the assay device from the initial compartment that T cells are added to, and the compartment containing the engrafted cancer cells. In this method cancer cells are not added to the same compartment and cancer cells are grown in 3D rather than 2D suspension.

Another assay is described within U.S. Pat. No. 11,613,7525 wherein cancer cells that have been previously engrafted in a bio scaffold in a separate culture system are transferred to a single chamber assay device and allowed to expand for a period of time, prior to the addition of T cells and/or the manufactured CGT Drug Product to the same chamber (compartment and chamber are used interchangeably). The assay device allows for the evaluation of Tumor killing of 3D engrafted tumor cells over a long period of time. The methods and devices described in the aforementioned abstract and the prior art require that cancer cells be engrafted in a 3D bio scaffold in a separate culture system, transfer of the bio scaffold containing engrafted cancer cells to the assay device, followed by the administration of T cells to the same chamber.

Neither U.S. Pat. No. 11,613,725 nor the previously referred to abstract (PMID: 24819215) go into detail about supplementation of bioluminescent substrate, however, skilled artisans recognize that all prior art methods require the supplementation of bioluminescent substrate on the day of bioluminescence measurement. There exists a need for simplified methods and devices that allow for potency assays that include multiple control conditions and a testing condition in duplicate, and do not require substrate supplementation or an initial step of tumor engraftment in a 3D bio scaffold followed by transfer of the 3D bio scaffold to the assay device.

The introduction of the G-Rex bioreactor device, U.S. Pat. No. 9,255,243, has eliminated much of the complexity and manual interventions associated with feeding cells media, and splitting cell cultures into new devices. With G-Rex, a culture no longer requires feeding and thus, simplified, and de-risked CGT manufacturing processes are now possible.

U.S. Pat. No. 11,613,725 builds upon the discoveries of U.S. Pat. No. 9,255,243 and discusses methods and devices that allow for improved long-term T cell product evaluations facilitated by novel device geometries that include multiple culture compartments with gas permeable growth surfaces and compartment wall heights that allow increased media volume to gas permeable cell growth surface area ratios. Furthermore, the disclosure includes compartment wall structures that allow fluid communication between adjacent compartments in a horizontal plane.

The methods and devices disclosed herein improve upon the methods and devices of U.S. Pat. No. 11,613,725 by providing novel methods and devices that do not require cancer cell engraftment into a bio scaffold and allow for direct measurement of cytotoxicity of thawed cryopreserved Cell Product assessed by direct addition of thawed cryopreserved Cell Product into the assay device, and a single addition of bioluminescence substrate. Furthermore, fluorescence can be stably integrated into cells for long-term assessment, and standard control target cell lines stably integrated can be developed for use in a CGT drug product's potency assay. In some embodiments, the devices disclosed herein are opaque, so as to limit the interference of the measured bioluminescent light measured in adjacent wells, greatly improving the accuracy of bioluminescence measurements.

In summary, a need exists for improved assay devices and methods that bring more efficiency, repeatability, and accuracy to potency assays allowing more reliable correlation of potency assay measurements with clinical effectiveness by characterizing cryopreserved CGT drug products in the state that they are formulated and as near as possible to the method of administration to patients. This is accomplished by the methods and devices disclosed herein by eliminating washes, buffer exchanges, or culturing of the drug product cells prior to use in this method. The methods and devices of this invention allow for the in vitro long-term characterization of cell product cytotoxicity using bioluminescent target cells without the need for addition of bioluminescent substrate beyond initial setup. Additionally, the devices and methods of this invention provide gas permeable cell culture devices with gas permeable growth surfaces with gas permeable cell growth surface area to media volume ratios aligned with commercially available G-Rex cell culture devices and methods for making devices with opaque walls to facilitate bioluminescent imaging.

### SUMMARY OF THE INVENTION

It has been discovered that a manufactured cell product, regardless of whether it is manufactured in research, process development, clinical or a commercial settings, can be evaluated in a novel long term manner within a gas permeable potency assay device by adding culture media, bioluminescent target cells such as cancer cells, substrate, and the cell product into the assay device. Bioluminescent target cells may be added to the assay device prior to the cell product, such as 1, 2, 3, 4, 5, 6, 7 or more days prior. There may be occasions where the bioluminescent target cells are added at about the same time as the cell product, such as less than one day or at the same time.

The geometry and design of the gas permeable assay device allows for an extended duration of cell growth, including the bioluminescent target cells and/or the cell product for a period of time beyond conventional potency assays without feeding, including 3, 4, 5, 6, 7, 8, 9, 10, 11, 12, 13, 14, 15, 16, 17, 18, 19, 20, 21, 22, 23, 24,25, 26, 27, 28, 29 and 30 days and anywhere in between. Unlike conventional assays for cell product, during the period of time when the cell product and the bioluminescent target cells are in the novel potency assay device, bioluminescence is measured without the need for addition of bioluminescence substrate or culture media to feed cells.

The novel potency assay also allows for the addition of cell product that has been cryopreserved and thawed, but not washed, to be added into the assay device. Thus, unlike prior assays where the cryopreserved cell product must be washed of the cryopreservation media after thaw, it has been discovered that the cell product need not be washed of the cryopreservation media, and the cell product inclusive of thawed cryopreservation media can be added into the assay device in which culture media resides. This has the advantage of simplifying the steps needed to conduct the assay.

Additionally, existing potency assays rest the cell product in culture media after they have been thawed. The novel assays eliminates the need for resting. This has the benefit of more closely mimicking the in vivo use of the manufactured CGT product, since it is delivered to the patient in the absence of a wash or a rest.

In clinical application the potency assay methods and devices of this invention allow for standardized potency assay test kit. The kit can include any combination of the elements present in the standard assay kit such as the assay device, the bioluminescent substrate, culture media, cytokines, target cells, and protocol for use.

The novel potency assay kit can be qualified for use in the Chemistry, Manufacturing, and Controls (CMC) section of an Investigative New Drug Application for various CGT products, such as CAR-T cells, and TCR, or any other CGT drug product known to those skilled in the art.

The potency assay devices and methods are a platform technology that can be standardized and used by various cell product developers and commercial cell product manufacturers without changing as the assay device, the bioluminescent substrate, culture media, cytokines, target cells, bioluminescent imaging equipment, and protocol for use. Such a platform may be well received by regulatory agencies such as the FDA because the same measuring stick will be applied to similar manufactured CGT products (i.e., CAR T, TIL, etc.) from company to company.

As a developmental tool, the assay devices and methods herein can be used to compare cell product candidates. For instance, several genetic engineering constructs can be compared to evaluate likelihood of clinical success thereby de-risking pre-clinical and clinical studies. Additionally, the cell product manufacturing processes and/or platforms can be compared by using the assay devices and methods disclosed herein for in vitro long-term characterization of cell product cytotoxicity from each manufacturing process and/or platform.

The assay devices and methods can facilitate cell product characterization and cell product candidate characterization. The methods and devices described herein allow for sufficient numbers of cells to be available for downstream analyses after completion of the long-term characterization assay. This allows any subsequent downstream data analyses to be directly correlated to the results of the assay. For example, cell product and/or cell product candidates, can be evaluated by other methods prior to initiating the assay and after completion of the assay. This allows the characterization of changes in the cell product that may have occurred during the assay. For example, at the end of any of the assay methods described herein, the cell suspension can be divided into cell product and target cell fractions for separate downstream analyses.

In other potency assays, like flow cytometry-based assays, killing is determined as an endpoint measurement and further downstream analysis is not possible. Additionally, flow cytometry-based assays are performed with low cell numbers, leaving a small quantity of cells samples available for further analysis. Similarly, microwell plate-based bioluminescence assays are performed with cell quantities that do not allow in-depth characterization analysis after assay execution.

Genomics technologies, at both bulk cell and single-cell levels, can be used in downstream analyses of effector cells (and/or target cells) to understand changes in gene expression (RNA-seq), changes in chromatin structure (ATAC-seq), changes in transcription factor binding (ChIP-seq), and/or immune cell profiling by TCR/BCR sequencing.

Proteomics technologies, at both bulk cell and single-cell levels, can be used in downstream analyses of effector cells (and/or target cells) to understand changes in the proteome (SDS-PAGE, Western blot, reverse phase protein arrays, mass spectrometry), surface protein expression (flow cytometry, CyTOF), and/or protein-protein interactions (co-immunoprecipitation).

Metabolomics technologies, at both bulk cell and single-cell levels, can be used in downstream analyses of cell product (and/or target cells) to understand metabolite changes (mass spectrometry, 13C metabolic flux analysis) and/or changes to cell glycolysis/oxidative phosphorylation pathways (Seahorse technology).

The present invention includes a method for evaluating cell product potency, the method comprising: adding bioluminescent target cells, media, and a bioluminescent substrate into at least one well of a gas permeable potency assay device comprising a housing comprised of at least one cell culture well with a gas permeable, liquid impermeable material, the inside surface of said gas permeable, liquid impermeable acts as a cell growth surface, and walls structured to allow media to reside at a media volume to gas permeable cell growth surface area ratio of at least 2 mL/cm2, wherein the housing allows ambient gas to contact the outside surface of said gas permeable, liquid impermeable material; thawing a cryopreserved cell product and, without washing, adding the cell product into at least one well containing the bioluminescent target cells; measuring a bioluminescent signal in the at least one well containing the cell product; and not adding media or bioluminescent substrate during a period of time beyond at least two days.

Further, the bioluminescent target cells may express luciferase or the bioluminescent target cells may express a fluorescent reporter. The walls of gas permeable potency assay device may be opaque. The cell product is CAR-T cells targeting CD19 and the bioluminescent target cells may be a bioluminescent cancer cell line expressing CD19. Further, the period of time for the method is up to 30 days. Alternatively, wherein the period of time for the method of time is 5 days.

### BRIEF DESCRIPTION OF THE DRAWINGS

FIG. 1 is an outline of some of the differences between the prior art and the methods and devices disclosed herein.

FIG. 2 is a gas permeable potency assay device with a lower gas permeable material configured to allow cells to distribute evenly about its lower surface and provide gas to the underside of the lower gas permeable material.

FIG. 3 is a gas permeable assay device configured in a multiple well format, capable of holding an increased volume of medium per well relative to traditional multiple well tissue culture devices.

FIG. 4A is an image of the bioluminescent signal captured five days after the drug product (CAR-T cells) was added to the gas permeable assay device as described in Example 1.

FIG. 4B is a graph displaying the fold change in target cell growth indicating the CAR-T cytotoxicity over a long period of time in the assay conditions described in Example 1.

FIG. 5A is a an image of the bioluminescent signal depicting the “Noise” measured in adjacent wells of devices with transparent walls (left) versus opaque walls (right).

FIG. 5B is a bar graph comparison of the average measured percentage of “Noise” measured in adjacent wells in the device with transparent walls compared to the device with opaque walls.

FIG. 6 is a graphical representation of the fold change in target cell growth as measured by the bioluminescent signal over time in the assay conditions described in Example 3.

FIG. 7 is a graphical representation of the fold change in target cell growth as measured by the bioluminescent signal over time in the assay conditions described in Example 4.

FIG. 8 is a graphical representation of the fold change in target cell growth as measured by the bioluminescent signal over time in the assay conditions described in Example 5

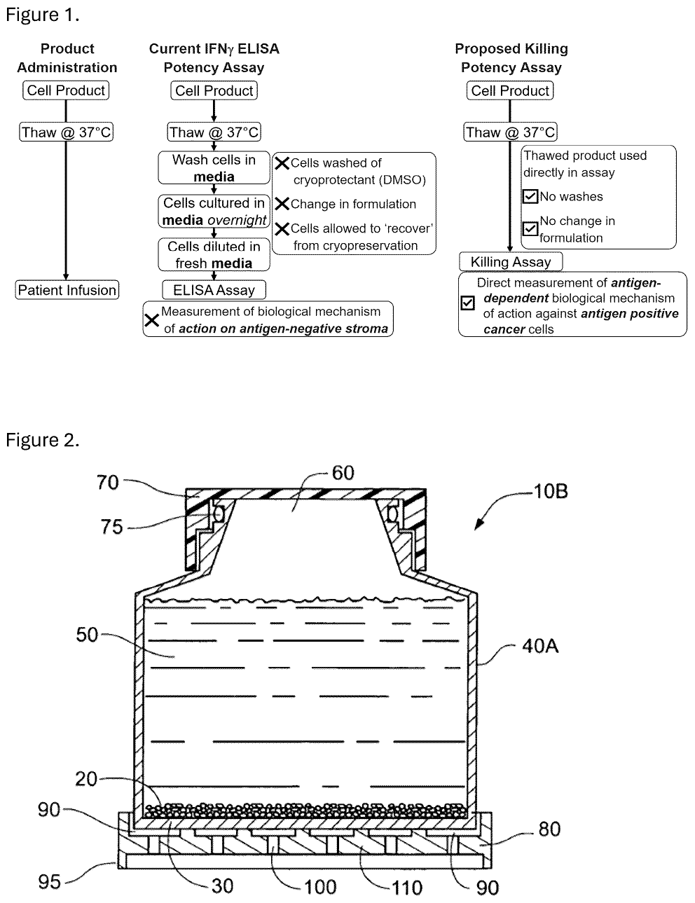

### DETAILED DESCRIPTION OF THE INVENTION

### Definitions

Cell Product refers to cells with a capacity to kill target cells.

Cryopreserved Cell Product refers to cells with a capacity to kill target cells that have been cryopreserved.

Thawed Cell Product refers to Cryopreserved Cell Product that has been thawed to a state where it is no longer frozen and the cryopreservation material is not separated from the Cell Product.

Target Cells refers to cells that are intended to be recognized and killed by the Cell Product.

### BRIEF DESCRIPTION OF THE DRAWINGS

Bioluminescent Target Cells refers to cells that are bioluminescent and that can be recognized and killed by the Cell Product.

Bioluminescent Signal refers to the detectable state of bioluminescence which is an indicator of the presence of Bioluminescent Target Cells. The Bioluminescent Signal will decrease as Target Cells are killed.

Bioluminescent Substrate refers to the presence of a molecule that produces light when it reacts with oxygen.

Media refers to a volume of liquid that provides cells with nourishment.

Negative Control are cells that can recognize the Target Cells but cannot kill the Target Cells.

Positive Control refers to cells that have a known capacity to kill the Target Cell.

Test Sample refers to the cell product that is being assessed for potency.

By operating gas permeable potency assay devices with media volume to growth surface area ratios that exceed what is possible in traditional assay devices, numerous benefits can accrue that have not been previously possible including the ability to perform long-term potency assays that are uninterrupted by human intervention, assess thawed cryopreserved cell product as it is formulated without need of separating the cells from the cryopreservation reagent, and allowing the long term characterization of cell product cytotoxicity without the need for repeated addition of a bioluminescent substrate.

The present invention includes gas permeable cell potency assay devices configured to function in a static mode. Many of the ideal structures and features of the gas permeable potency assay devices are described in U.S. Pat. No. 9,255,243, which is hereby incorporated by reference in its entirety.

Skilled artisans are highly encouraged to recognize that use of the word “embodiment” herein does not restrict any element described within a given embodiment from being used within another embodiment. Every element within a described embodiment is not inextricably linked to that particular embodiment. With that in mind, the artisan is encouraged to mix and match any combination of the cumulative elements found in the embodiments to perform long term assays to identify the specific characteristics of a cell product that the artisan seeks to assess, including but not limited to cell product capacity to kill target cells, inclusion of controls, use of media in quantities that will allow undisturbed assay conditions, ratios of media volume to cryoprotect volume that will avoid need of separating cells within the thawed cell product from cryoprotectant,

The potency assay device can comprise a housing with at least one open well, each well having a bottom comprised of gas permeable material, the bottom comprised of gas permeable material being a gas permeable cell growth surface, and each well having a preferred internal media volume to gas permeable cell growth surface area ratio of at least 2 mL/cm2. The shape of the wells can be square, rectangular, and/or cylindrical.

The gas permeable potency assay device can comprise multiple separate wells, each well including walls structured such that the media volume to gas permeable cell growth surface area of each well is equivalent and is greater than 2 mL/cm2 of gas permeable cell growth surface area. Walls can be comprised of any biocompatible material and should mate to lower gas permeable material in a manner that forms a liquid tight seal. The methods of mating a lower gas permeable material to walls include adhesive bonding, heat sealing, compression squeeze, and any other method commonly used for generating seals between parts. As an option, walls and lower gas permeable material can be formed of the same material and fabricated as a single entity. For example, if silicone is used, walls and the lower gas permeable material could be liquid injection molded, or dip molded, into a single gas permeable piece. Walls are preferably configured with enough structural strength that medium is retained in a relatively symmetrical shape above gas permeable material in order to make most efficient use of lab space, minimize gradient formation within a medium, and allow a uniform deposit of cells.

It is beneficial if ambient gas can make relatively unobstructed contact with the underside of the lower gas permeable material. For example, in incubators in which the shelves are non-perforated, gas transfer in and out of the culture can be limited if the lower gas permeable material makes contact with the incubator shelf. In the embodiment shown in the cross-sectional view of FIG. 2, which depicts a gas permeable device having a single well, lower gas permeable material support 80 acts to ensure that lower gas permeable material 30 is in contact with ambient gas by maintaining a gas compartment 90. In the preferred embodiment, gas compartment 90 is maintained by allowing lower gas permeable material support 80 to make partial contact with lower gas permeable material 30 in a manner that does not diminish the amount of gas exchange required to support the culture. In addition to allowing exposure to ambient gas, lower gas permeable material support 80 maintains lower gas permeable material 30 in a substantially horizontal state such that cells 20 do not pile up in any low points. That would cause diffusional gradients and limit cell growth relative to a condition in which cells 20 could distribute evenly across lower gas permeable material 30. Therefore, a design objective for lower gas permeable material support 80 may be to contact lower gas permeable material 30 in as many locations as needed to keep it substantially horizontal while still allowing adequate gas contact with the lower surface of lower gas permeable material 30. Those skilled in the art will recognize there are many ways to achieve this objective. As shown in FIG. 2, projections 110 achieve this objective.

A “bed of nails” configuration is one way to maintain lower gas permeable material 30 in a substantially horizontal position while allowing adequate gas exchange. As also shown in FIG. 2, lower gas access openings 100 allow gas to enter and exit gas compartment 90 of lower gas permeable material support 80 by passive diffusion. This allows gas permeable cell culture device 10B to function in ambient conditions without need of ancillary pumping mechanisms. Feet 95 elevate lower gas permeable material support 80, allowing ambient gas to be available to lower gas access openings 100. This information also is applicable to maintaining a gas compartment around sidewalls when the device functions as described on its side in either the rolling or non-rolling mode. Other possibilities of allowing adequate gas access to a gas permeable material can be utilized. For example, the CELLine™ products from Integra Biosciences AG utilize open mesh elevated from a lower plastic support by feet to allow gas access to the gas permeable membrane. U.S. Pat. No. 5,693,537 also provides additional guidance for this feature.

The potency assay device can be comprised of multiple separate wells, each including a gas permeable growth surface and walls structured such that the media volume to gas permeable cell growth surface area of each well is equivalent and is preferably from 2 mL/cm2 and 20 mL/cm2 of gas permeable cell growth surface area, including 2.1, 2.2, 2.3, 9.8,9.9, and 20 mL/cm2.

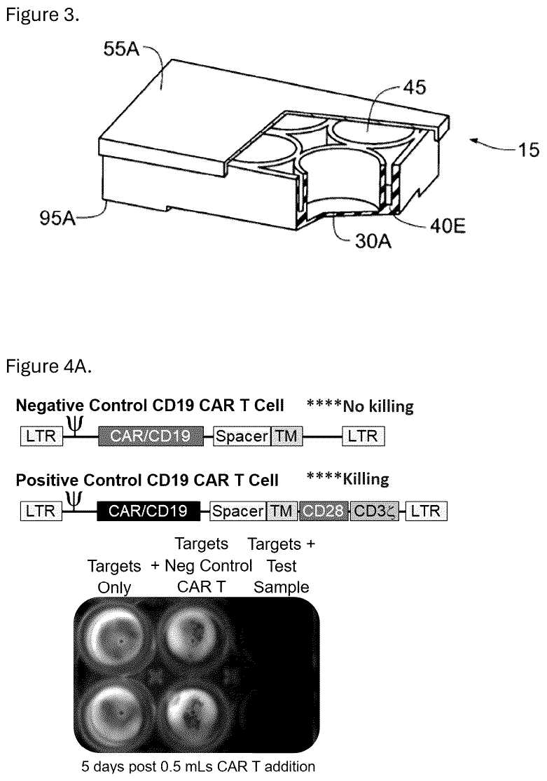

As described in U.S. Pat. No. 9,255,243, and shown in FIG. 3, gas permeable potency assay devices can be configured as multiple well plate 15, in which the bottom of each well is gas permeable and is a cell growth surface. Although a six well plate is shown, any number of individual wells 45 can be present, including the traditional formats of six, twenty-four, forty-eight, and ninety-six wells, or non-traditional formats including two, four, three and other non-traditional formats. Walls 40E are structured to allow medium to reside at a height above lower gas permeable material 30A that exceeds the wall height of traditional multiple well plates, thereby increasing the number of cells that can reside in each well while reducing the footprint relative to traditional multiple well plates. Lower gas permeable material 30A can be supported in a substantially horizontal position as shown in FIG. 2, where lower gas permeable material support 80 is configured with lower gas access openings 100 in communication with gas compartment 90. In the event that the span of the bottom of well 45 is small, support may be unnecessary because the physical strength of lower gas permeable material 30A can retain it in an adequate horizontal position, depending on the thickness and physical properties of the gas permeable material. In this case, feet 95A can be used to elevate gas permeable multiple well plate 15 so that gas transfer is not a problem in an incubator with non-perforated shelves. Top cover 55A prevents contamination and minimizes evaporation.

The potency assay device can be comprised of six separate wells, allowing for two separate control measurements and a test measurement each in duplicate, each well having a bottom comprised of gas permeable material, the bottom comprised of gas permeable material being a gas permeable cell growth surface and structured such that the media volume to gas permeable cell growth surface area of each well is equivalent and is preferably between 2 mL/cm2 and up to 20 mL/cm2 of gas permeable cell growth surface area, including 2.1, 2.2, 2.3, 2.4, 2.5, 2.6, 2.7, and increments of 0.1 mL/cm2 up to and including 20 mL/cm2 or any increment less than 0.1 mL/cm2.

The gas permeable cell growth surface of the potency assay device can be comprised of silicone.

Any area of the potency assay device can be made opaque by use of tints and colors such as green or black.

When the potency assay device includes multiple wells a top cover can be clear or opaque device.

Those skilled in the art will recognize that the gas permeable material should be selected based on a variety of characteristics including gas permeability, moisture vapor transmission, capacity to be altered for desired cell interaction with cells, optical clarity, physical strength, and the like. Silicone is often a good choice.

In some embodiments a cryopreserved cell product, such as a CAR-T or TCR cell product is thawed and added directly to target cells that are being cultured in the gas permeable potency assay device.

The methods for using a multiwell potency assay device can comprise introduction of media, reagents, bioluminescent target cells, substrates such as luciferin and thawed cell product into one or more of the wells such that a comparison of bioluminescent between well(s) that have cell product and cells that do not have cell product can be made.

The methods for using a multiwell potency gas permeable assay device may include:
adding thawed cell product, a quantity of bioluminescent target cells, substrate and media into a gas permeable assay device and allowing a period of incubation time for the cell product to diminish the quantity of bioluminescent target cells and
periodically assessing the magnitude of the bioluminescent signal at any given period of time such as increments of 16, 24, 36, 48, 60, 72, or more hours which may include 30 days or more and any time interval.

The methods for using a multiwell potency assay device may include:
adding thawed cell product, a quantity of bioluminescent target cells, substrate and media into a gas permeable assay device and allowing a period of incubation time for the cell product to diminish the quantity of bioluminescent target cells and
wherein the ratio of the volume of thawed cell product to media is from 0.001% to 30% and any increment in between.

The gas permeable portion of the gas permeable assay device may comprise silicone and in a preferred embodiment the silicone has a thickness of 0.004 to 0.033 inches and any increment in between.

Comparison of the bioluminescent light from any given well of the cell culture wells containing only bioluminescent target cells, is compared to the bioluminescence of the cell culture wells containing bioluminescent target cells and the cell product at multiple time points. The multiple time points can, for example, be at days 1, 2, 3, 4, and 5 and bioluminescence substrate does not need to be added to the assay device during the test. Skilled artisans recognize that the target cells will be cell product specific, in other words, the target cells used will depend upon the cell product being evaluated.

In some embodiments methods for using standardized media, reagents and an assay device are provided wherein bioluminescent target cells and standardized media and reagents are introduced into multiple cell culture wells of the assay device, allowed to grow in the standardized media and reagents for a period of time, and thawed cryopreserved cell product are added to at least one well of the multiple wells containing bioluminescent target cells, wherein the bioluminescence of the wells containing only bioluminescent target cells, is compared to the bioluminescence of the wells containing bioluminescent target cells and the cell product, at multiple time points. The multiple time points can be, for example, at days 1, 2, 3, 4, and/or 5, or any discrete time point therebetween sufficient to allow the cell product, to initiate killing of bioluminescent target cells. Bioluminescence substrate is added when the bioluminescent target cells and media area added to the assay device and does not need to be added to the assay device prior to measurement timepoints or during the assay. Skilled artisans recognize that the bioluminescent target cells will be cell product specific, in other words, the target cells used will depend upon the cell product being evaluated. Skilled artisans recognize there are several ways to enable real time assessment of growth by bioluminescent imaging and several ways to create bioluminescent target cells. For instance, any cancer cell line known to those skilled in the art to express a particular target of interest can by transduced with a with a retroviral vector encoding the fusion protein Fluc/eGFP (Firefly luciferase/enhanced green fluorescent protein). Transduction efficiency can then be determined 72 hours later via GFP expression (flow cytometry). After confirming transduction, single cell clones will be selected using a BD FACSAria II cell sorter or similar, and subsequently expanded for future testing.

In some embodiments methods for using media, reagents, and a gas permeable potency assay device in a multi well format 6 wells are undertaken wherein
a) bioluminescent target cells and media and reagents are introduced into six cell culture wells of the potency assay device,
b) allowed to grow in the standardized media and reagents for a period of time, and
c) thawed cryopreserved cell product is added to two wells of the six wells containing bioluminescent target cells; and control cells are added to two different wells of the six wells, and two containing only bioluminescent target cells are allowed to continue growing without interference, wherein the bioluminescence of the cell culture wells containing only bioluminescent target cells, is compared to the bioluminescence of the cell culture wells containing bioluminescent target cells and cell product and the cell culture wells containing bioluminescent target cells and control cells, at multiple time points. The multiple time points can be up to 30 days or more, and preferably with 20 days and more preferably with 10 days, or any time point there between sufficient to allow cell product to initiate killing of target cells. and bioluminescence substrate does not need to be added to the assay device throughout the test. Skilled artisans recognize that the target cells will be cell product specific. Similarly, skilled artisans recognize that control cells may be cell product specific.

### EXAMPLES

### Example 1-Assessing the cytotoxicity of thawed cryopreserved manufactured cell product.

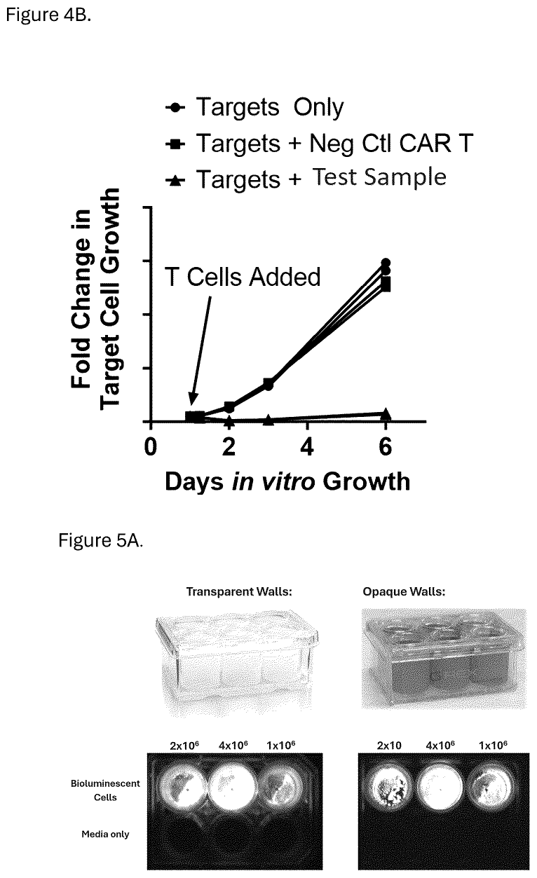

Cytotoxicity of thawed cryopreserved cell product is assessed by direct addition into the gas permeable potency assay device which contains target cells. This non-limiting example demonstrates that when a cryopreserved cell product, such as CAR-T cells, is thawed, it can be added directly to target cells cultured in gas permeable potency assay device containing at least 4 mLs of media per cm2 of gas permeable cell growth surface area. When added directly to target cells cultured in the gas permeable potency assay device, thawed cryopreserved CAR-T cell product is able to eliminate target cell numbers compared to target only control and negative control CAR-T cell product (FIG. 4A and FIG. 4B). For this assay, on day 0, 2×10⁶ bioluminescent target cells were added to an assay device containing culture media and 5 μM D-Luciferin luciferase substrate. On day 1 of the assay, CAR-T cell products that had been cryopreserved in CS10 media at 10×106 cells/mL were thawed and added directly into wells of the assay device containing target cells. For this assay, two different types of CAR-T cell products were added: 1) a test sample comprised of a second generation CD19 CAR constructed with a CD28 co-stimulatory domain capable of the specific elimination of CD19 positive target cells, and 2) a Negative Control sample comprised of a CD19 CAR that lacks the CD3 zeta intracellular signaling domain, rendering it incapable of specific elimination of CD19 positive target cells. Target cell bioluminescence of each well containing either test sample, negative control sample, or targets along was measured daily without disruption of the sample interaction with target cells using a Bio-Rad ChemiDoc instrument. As shown, cryopreserved CAR-T product (test sample) eliminates bioluminescent target cells, which can be measured by the difference in the bioluminescent signal in the Targets+Test Sample wells compared to the Targets+Negative Control CAR-T and the Targets Only wells 5 days after initiation of the assay (FIG. 4A). Time-course quantitative bioluminescence measurements are indicated in FIG. 4B, the fold change in target cell growth is measured by the change in the bioluminescent signal. As shown, target cell bioluminescence increases over time, but is controlled in the test sample wells. While FIG. 4B indicates the cytotoxicity of the Test Sample, in this case a 2nd Generation CAR-T over time. Skilled artisans will recognize that cytotoxicity need not be measured frequently and can be measured at a particular time. Stated differently, cytotoxicity of the CAR-T Test Sample can also be calculated as an endpoint measurement. Here, cytotoxicity can be represented as the percent reduction in target cell bioluminescence when in contact with test samples versus target cells alone: (1−[(test sample bioluminescence)/(targets alone bioluminescence)])*100. Cytotoxicity of the 2nd Generation CAR T cell product in this example was calculated to be 96% compared to 21% non-specific cytotoxicity of the negative control CAR T cell product.

These data confirm that this in vitro cytotoxicity assay can directly and specifically assess cryopreserved cell therapy products without washing the cell therapy product prior to use in the assay. Stated differently, the cell therapy product can be thawed and directly added to the target cells. This method of cytotoxicity measurement is largely unaffected by the cryopreservation formulation, as indicated by the lack of change in target bioluminescence in the negative control CAR-T cell product at early assay timepoints.

Skilled artisans will recognize the specific seeding density of the bioluminescent target cells, the device surface area to media volume ratio, the exact amount and type of bioluminescent substrate added, the type of media that the CAR T cells were cryopreserved in and the concentration at which they were cryopreserved, the specific CAR-T cells evaluated, the specific control cells evaluated, and the type of equipment used to measure bioluminescence are not inextricably linked to the examples described herein and therefore various elements can be extracted from the examples herein and combined with other elements described in the examples and throughout the disclosure. Example 1 merely provides an example of one potential use of the methods and devices described herein and it is recognized that CAR-T cells targeting antigens other than CD19 such as CD20, Mesothelin, BCMA, CD22, CD138, PSMA, Biotin, EGFRvIII, HER2, MUC1, NKG2D and other antigens known to those skilled in the art can be evaluated using this potency assay. Also, TIL products, TCRs, TAAs, VST's, and others can be evaluated using this potency assay. The number of duplicates in the potency assay methods can be increased or decreased, and the length of the potency assay can be increased or decreased and the number of bioluminescent signal measurements and the duration of time/incubation of cells in between bioluminescent signal measurements can be varied.

### Example 2

### An Opaque Gas Permeable Assay Device Format Can Significantly Reduce Light “Spillage” Out of Wells

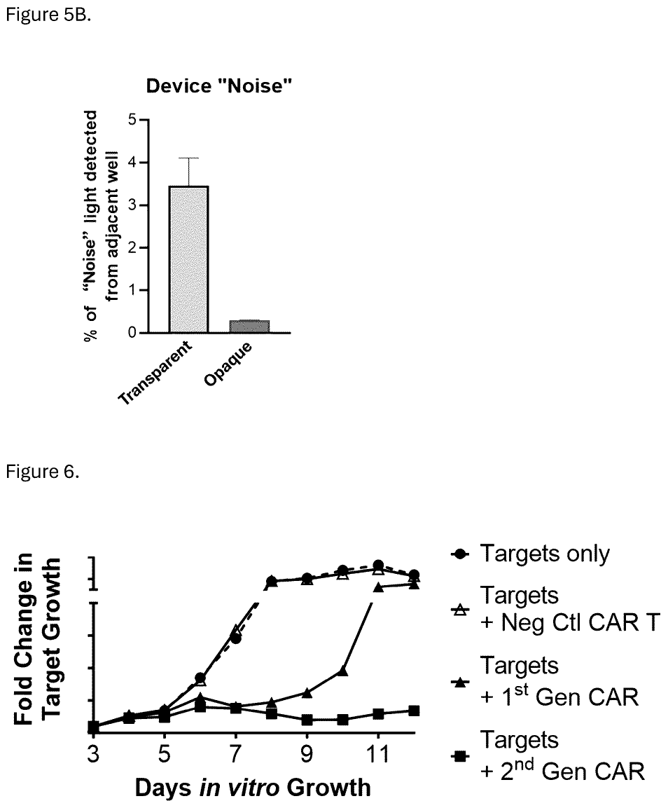

In this non-limiting example, one of the potency assay devices included wells with transparent walls and the other potency assay device included wells with opaque walls. To determine if there is a difference in bioluminescent “Noise” measured in adjacent wells, three wells were seeded with either 1×10⁶, 2×10⁶, or 4×10⁶ bioluminescent cells and cultured in each device, while the other three wells in each device were filled with media only, as depicted in FIG. 5A. The bioluminescent light detected in all wells was quantitated using a Bio-Rad ChemiDoc instrument. The average amount of bioluminescent light measured in each of the three wells containing media only, was compared to the average amount of light measured in the wells containing bioluminescent cells. As can be seen in FIG. 5A, the amount of bioluminescent “Noise” measured in adjacent wells is greater in the assay device having wells with transparent wells. As shown in FIG. 5B, the average amount of bioluminescent light measured in wells adjacent to the wells containing bioluminescent cells was 3.4% of the bioluminescence measured in the wells containing bioluminescent cells. However, in the assay device having opaque walls, the average amount of bioluminescent light measured in wells adjacent to bioluminescent cells was significantly reduced to 0.29%. These data demonstrate that the opaque format will considerably reduce the ability of light emitted from bioluminescent cells in adjacent wells to interfere with accurate measurements.

Skilled artisans will recognize that there are several ways to reduce bioluminescent “Noise”. The walls of the wells can be any color that would reduce “Noise” including green, black, blue, red, purple, teal etc., Skilled artisans will also recognize that reducing or eliminating bioluminescent “Noise” is not required and the assay methods described herein can be executed using assay devices including wells with transparent or near transparent walls or wells that are opaque, or of any other color.

### Example 3

### The Potency Assays and Methods Can be Used to Differentiate Superior Cell Products

The enhanced cytotoxicity and proliferation of 2nd generation CAR T cells compared to 1st generation CAR T cells have significantly improved clinical outcomes (PMID: 11714771, 9743337). When tested using the methods and devices described herein, 1st generation CAR T cells were indistinguishable from 2nd generation CAR T cells in their ability to control lymphoma cell growth through a short, one week assay timeframe. However, when the assay time is extended, the 2nd generation CAR-T is better able to control lymphoma cell growth compared to 1st gen CAR-T, where lymphoma cell growth surges. As shown in FIG. 6, the fold change in target cell growth as measured by the bioluminescent signal, is controlled by the 2nd generation CAR-T cells whereas, the 1st generation CAR-T cells are unable to control lymphoma cell growth beyond 7 days.

Skilled artisans will recognize that any cell product can be evaluated using the methods described in non-limiting Example 3. The cells can be CAR-T cells, TCR cells, NK cells, antigen specific T cells, gamma-delta T cells, CAR-NK cells, and the like, and the bioluminescent target cells can be any cancer cell line known to those skilled in the art. The length of the assay can proceed for at least up to 16 hours, 24 hours, 36 hours, 48 hours, 60 hours, 72 hours, 96 hours, 120 hours, 144 hours, 168, 192 hours, 216 hours, and up to two weeks and beyond. The frequency of measurement can vary. Skilled artisans will recognize it is important to obtain measurements of bioluminescence to establish a trend and monitor the progress of the assay, however, it is recognized that defined measurement timepoints and frequencies are not essential to the execution of the assay.

### Example 4

### Potency is Correlated With Identity/Purity of CAR T Cell Product (Percentage of CAR-Transduced T Cells)

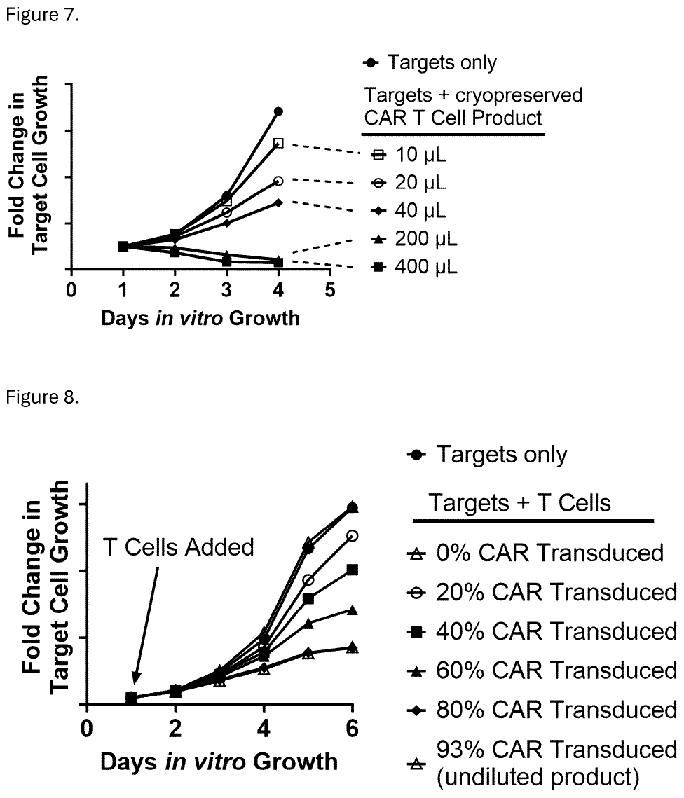

This non-limiting example shows how potency can be determined with as little as 20-40 μL of cryopreserved product. In this example, a potency assay device having six wells, a G-Rex 6 well plate (www.wilsonwolf.com catalogue 80240M) was seeded with bioluminescent target cells and the bioluminescent target cells were cultured in each well. A CAR-T cell drug product cryopreserved in CS10 was thawed and without washing or resting the thawed CAR-T cell drug product, one well of the G-Rex 6 well was seeded with 10 μL of thawed CAR-T cell drug product, and each of four other cells was seeded with 20 μL, 40 μL, 200 μL, and 400 μL respectively. As shown in FIG. 7, the fold change in bioluminescent target cell growth as measured by the bioluminescent signal is dependent upon the volume of cryopreserved cell product added to the bioluminescent target cells, thus the potency assay distinguishes between doses of cryopreserved Cell product.

The assay methods and devices described herein demonstrate that potency as measured by the methods described herein is correlated with identity/purity of CAR T cell product (percentage of CAR-transduced T cells). In this example, donor T cells were obtained and CAR T cells were manufactured and cryopreserved. T cells from the same donor with manufactured without introduction of the CAR by standard cell expansion methodologies known to those skilled in the art and cryopreserved. The Cryopreserved CAR and non-CAR T cells were thawed and mixed to represent varied CAR-T cell transduction efficiencies. The thawed cells were then added to the assay device and the fold change in bioluminescent target cell growth as measured by the bioluminescent signal was compared between the various represented CAR-T cell transduction efficiencies. As shown in FIG. 7, the percentage of transduction efficiency is correlated to the potency or cytotoxicity of the added effector cells (CAR-T cells) with higher representative transduction efficiencies being associated with lower fold expansion of bioluminescent target cells.

The devices and methods disclosed herein allow a novel potency assay comprising adding a quantity of media, bioluminescent target cells, and bioluminescent substrate into a gas permeable assay device and allowing a period of time for the bioluminescent target cells to establish growth as measured by the bioluminescent signal by placing the gas permeable assay device into a standard cell culture incubator. The gas permeable assay device may include multiple wells and the quantity of media, bioluminescent target cells, and substrate may be added to each of the multiple wells at the same time. The period of time for the Bioluminescent target cells to establish growth can be any period of time known to those skilled in the art that will allow the bioluminescent signal of the bioluminescent target cells to be measured. Alternatively, a quantity of media, bioluminescent target cells, cell product, and substrate can be added into the gas permeable assay device and placed in a standard cell culture incubator to incubate for a period of time. The gas permeable assay device may include multiple wells and the quantity of media, bioluminescent target cells, and substrate may be added to each of the multiple wells at the same time, while the cell product and any controls are added to only a portion of the wells. After a period of time, the gas permeable assay device is removed from incubation and the bioluminescent signal of the bioluminescent target cells is measured.

The period of time for the gas permeable assay device to remain in incubation is at least 16 hours (vs. 16 hours or less), 24 hours, 36 hours, 48 hours, 60 hours, 72 hours, at least 30 days and any time period therebetween. After the magnitude of the bioluminescent signal is measured without disturbing the cells with a pipette or any other cell culturing tool known to those skilled in the art, the gas permeable assay device may be placed back into a standard cell culture incubator for a period of time. After the period of time, the gas permeable assay device can then be removed from incubation and the bioluminescent signal can be measured again. The number of measurements is not limited. A diminished bioluminescent signal signifies cell product efficacy in killing the bioluminescent target cells.

Those skilled in the art will recognize that numerous modifications to the methods and devices described herein can be made thereof without departing from the spirit of the invention. Therefore, it is not intended to limit the breadth of the invention to the embodiments illustrated and described. Rather, the scope of the invention is to be interpreted by the appended claims and their equivalents. Each publication, patent, patent application, and reference cited herein is hereby incorporated herein by reference.

What is claimed is:

1. A method for evaluating cell product potency, the method comprising:
adding bioluminescent target cells, media, and a bioluminescent substrate into at least one well of a gas permeable potency assay device comprising a housing comprised of at least one cell culture well with a gas permeable, liquid impermeable material, the inside surface of said gas permeable, liquid impermeable acts as a cell growth surface, and walls structured to allow media to reside at a media volume to gas permeable cell growth surface area ratio of at least 2 mL/cm2, wherein the housing allows ambient gas to contact the outside surface of said gas permeable, liquid impermeable material;
thawing a cryopreserved cell product and, without washing, adding the cell product into at least one well containing the bioluminescent target cells;
measuring a bioluminescent signal in the at least one well containing the cell product; and
not adding media or bioluminescent substrate during a period of time beyond at least two days.

2. The method of claim 1, wherein the bioluminescent target cells express luciferase.

3. The method of claim 1, wherein the bioluminescent target cells express a fluorescent reporter.

4. The method of claim 1, wherein the walls of gas permeable potency assay device are opaque.

5. The method of claim 1, wherein the cell product is CAR-T cells targeting CD19 and the bioluminescent target cells are a bioluminescent cancer cell line expressing CD19.

6. The method of claim 1, wherein the period of time is up to 30 days.

7. The method of claim 1, wherein the period of time is 5 days.
